(19) United States
(12) Patent Application Publication
(10) Pub. No.: US 2009/0151728 A1
(43) Pub. Date: Jun. 18, 2009

(54) Respiratory Protection Device

(76) Inventors: Alison Kay McConnell, Bournemouth (GB); Johannes Alexander Paulus Paul, Pulham St. Mary (GB); Owen James Warren Evans, Brighton (GB)

Correspondence Address: Law Office of Ira S. Dorman, 330 Roberts Street, Suite 200, East Hartford, CT, 06108, US

(21) Appl. No.: 12/083,143

(22) PCT Filed: Oct. 4, 2006
(86) PCT No.: PCT/GB2006/003681

§ 371 Date: Feb. 4, 2009

(30) Foreign Application Priority Data
Oct. 11, 2005 (GB) 0520614.9

(51) Int. Cl. A61M 16/06; A62B 23/00

(52) U.S. Cl. 128/206.19; 128/206.24

(57) ABSTRACT

A respiratory protection device comprises a housing (1) defining a chamber within the housing. The chamber is open in a region which in use is adapted to be nearest to a user. A porous element (3) is located within the chamber and a communicating member (5) communicates with the porous element (3) within the chamber.

This invention relates to a respiratory protection device for personal use.

It is known to use face masks in order to provide protection from air pollution, and from smoke and dust inhalation. Particular users include commuters walking, jogging or cycling in cities, workers in dusty environments, as well as people engaged in self-rescue from smoke-filled environments. Known face masks suffer from a number of problems. For example, they cover the user's nose and can therefore be uncomfortable and make swallowing difficult. Although the area of a filter of a face mask may be considerable, a large proportion of the filter is in contact with the user's face to ensure a close fit for the mask and is therefore ineffective; the remaining surface area is therefore limited, leading to high flow resistance. In addition, the close fit to the face can cause irritation to the skin and local overheating of the face, which causes discomfort, acne and an increase in the effort sensation associated with physical activity.

A problem associated with physical activity in around 10 percent of the population is exercise-induced asthma. It is known that this is caused by drying of the lung airway lining, which triggers constriction of the airways. It is also known that humid environments attenuate the asthma response to physical activity. However, it is not been previously known how inhaled air can be humidified during physical activity in an acceptable, comfortable and effective manner. Therefore, there is a need for a respiratory protection device which humidifies inhaled air in a manner which is discrete, does not restrict airflow and which provides adequate humidification of the inhaled air to prevent the asthmatic response.

It is therefore an object of the present invention to provide a respiratory protection device which overcomes or ameliorates at least some of the above-described disadvantages.

According to the present invention there is provided a respiratory protection device comprising:

a housing defining a chamber within the housing, the chamber being open in a region which in use is adapted to be nearest to a user;
a porous element within the chamber; and
a communicating member communicating with the porous element within the chamber.

Thus, the device according to the invention allows a user to draw air into the user's body through the porous element in the chamber of the housing.

The porous element may be a foam material, preferably a polymeric open cell foam material.

The porous element may incorporate a hygroscopic material.

A filter may be incorporated in the porous element. The filter may comprise a web of filter material mounted on the porous element. In addition, the filter may comprise a layer of activated carbon material.

The communicating member may be provided with a plate-like portion adapted to be received in the mouth of the user, for example, behind the lips and in front of the teeth of the user. Alternatively, the communicating member may be provided with a mask-like portion adapted to be worn over the mouth of the user.

The communicating member may be provided with a tubular element adapted to communicate with the porous element.

The tubular element may be provided with a portion having gaps through an annular wall. The total area of the gaps may be greater, for example substantially 20 percent greater, than a cross-sectional area of the tubular element defined by the annular wall.

The communicating member may be provided with a wall portion adapted to retain a portion of the porous element within the chamber.

The wall portion of the communicating member may be provided with means, for example shoulders, adapted to secure the wall portion of the communicating member to securing members of the housing. The securing members of the housing may be of resilient material adapted to fit over the shoulders of the wall portion of the communicating member thereby securing the member to the housing.

The housing may be provided with first and second opposing surfaces.

An outlet may be provided in the first surface of the housing. A non-return valve may be mounted across the outlet in the first surface. The non-return valve may comprise a flap valve.

The outlet may be substantially coaxial with the communicating member. The cross-sectional area of the outlet may correspond substantially to the cross-sectional area of the tubular element of the communicating member.

A plurality of apertures may be provided in the first surface of the housing adapted to permit air to pass through the first surface during inhalation and exhalation by the user.

A retaining member, for example an extending lip, may be provided on the second surface of the housing adapted to retain the porous element within the chamber of the housing.

The chamber may extend laterally to one or both sides of the communicating member.

The housing may be curved so as in use to extend around the face of the user.

For a better understanding of the present invention and to show more clearly how it may be carried into effect reference will now be made, by way of example, to the accompanying drawings in which:

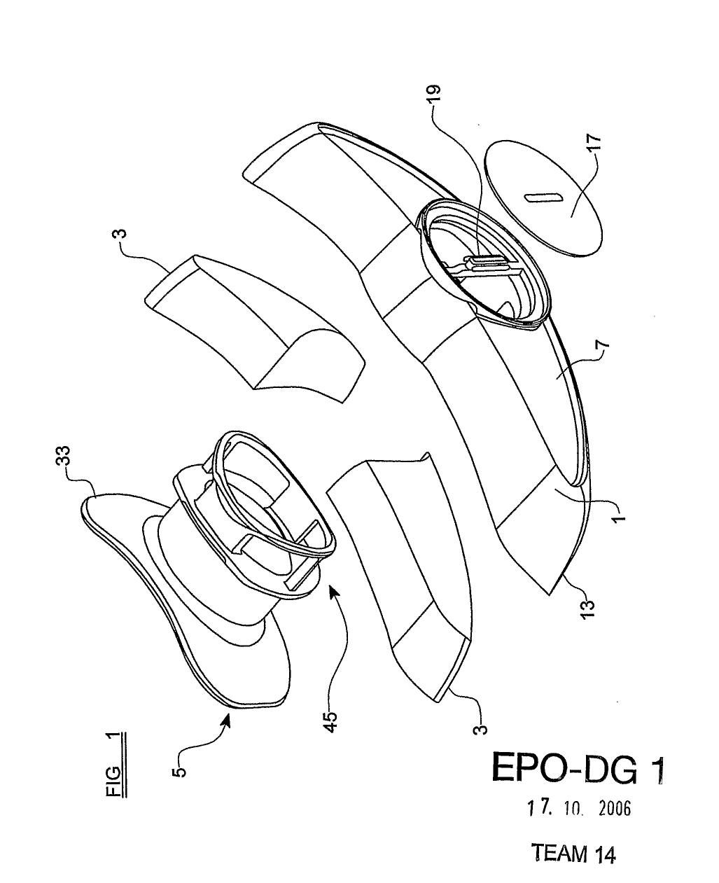

FIG. 1 is an exploded front perspective view of a first embodiment of a respiratory protection device according to the present invention;

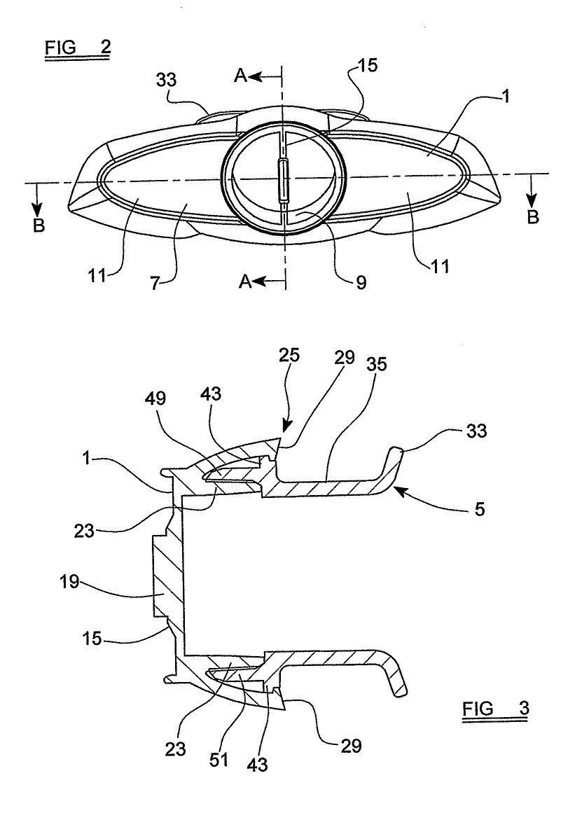

FIG. 2 is a front view of the respiratory protection device shown in FIG. 1 with a flap valve removed;

FIG. 3 is a cross-sectional view along the line A-A of the respiratory protection device shown in FIG. 2;

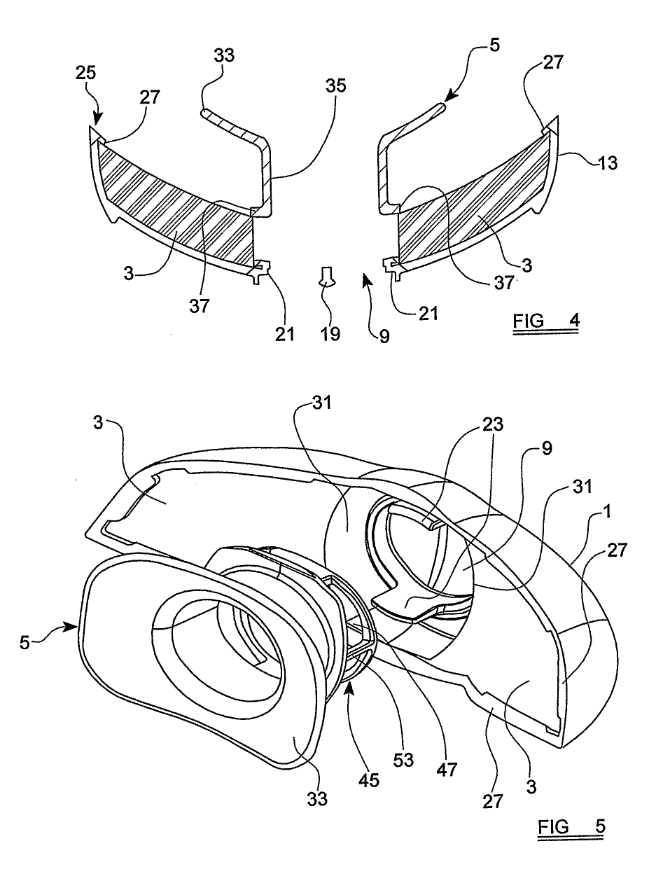

FIG. 4 is a cross-sectional view along the line B-B of the respiratory protection device shown in FIG. 2;

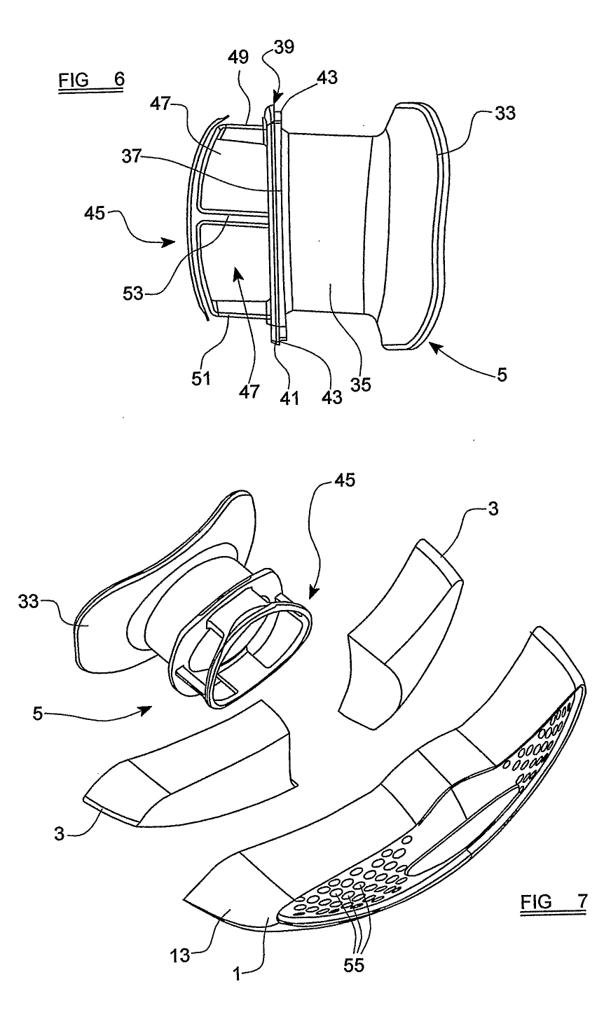

FIG. 5 is an exploded rear perspective view of the respiratory protection device shown in FIG. 1 with a flap valve removed;

FIG. 6 is a side view of a first embodiment of a communicating member of the respiratory protection device shown in FIG. 1;

FIG. 7 is an exploded front perspective view of a second embodiment of a respiratory protection device according to the present invention;

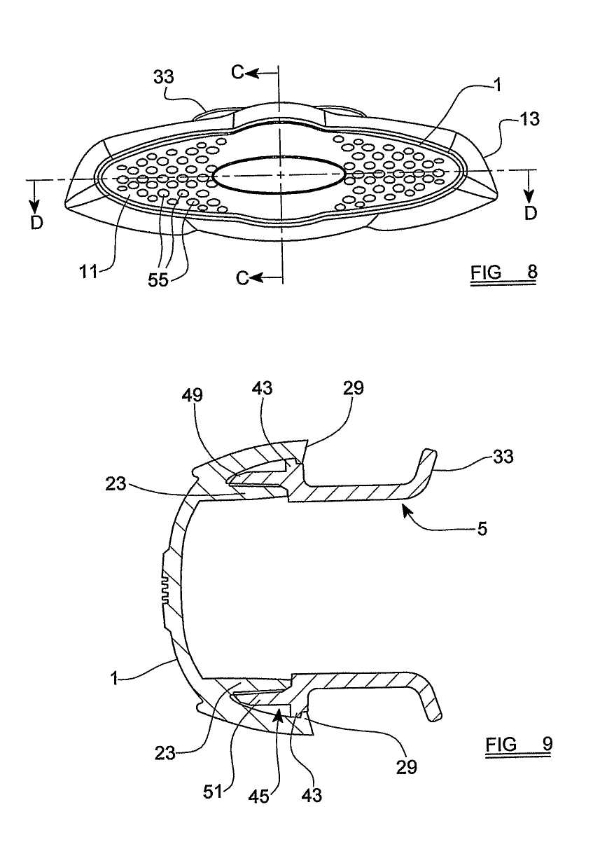

FIG. 8 is a front view of the respiratory protection device shown in FIG. 7;

FIG. 9 is a cross-sectional view along the line C-C of the respiratory protection device shown in FIG. 8;

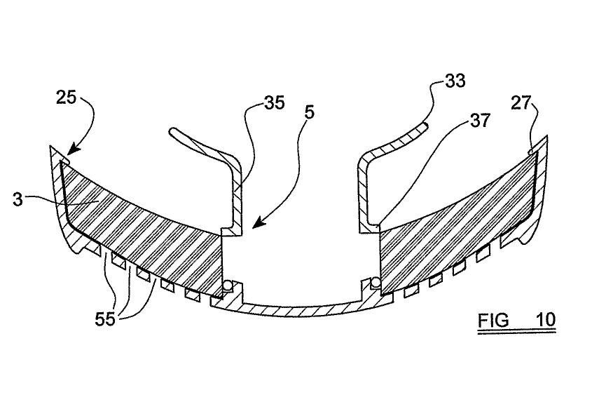

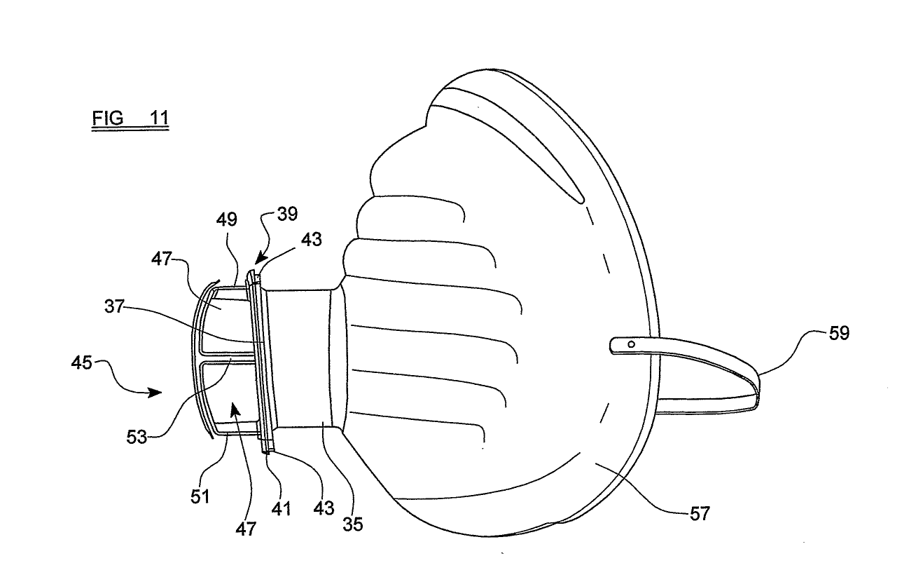

FIG. 10 is a cross-sectional view along the line D-D of the respiratory protection device shown in FIG. 8; and

FIG. 11 is a side view of a second embodiment of a communicating member of a respiratory protection device in accordance with the present invention.

FIGS. 1 to 5 show a first embodiment of a respiratory protection device in accordance with the present invention comprising a housing 1, a pair of porous foam elements 3 and a communicating member in the form of a mouthpiece 5.

The housing 1 is in the form of a chamber which is generally U-shaped in cross-section forming top and bottom walls of the housing with a (first) forward surface 7 therebetween to resist the ingress of rain or the like into the device. An outlet 9 with an elliptical cross-section, the major axis of the ellipse being substantially horizontal, is provided in a central region of the forward surface of the housing 1 (see FIG. 2). A pair of wings 11 extend laterally, one from each side of the central region of the forward surface of the housing, forming a first side portion and a second side portion of the chamber. Each wing 11 is curved so as to extend in use around the face of the user and the outer ends of the wings are closed at 13 forming side walls of the chamber.

It should be noted the terms “forward”, “rear”, “upper”, “lower” and the like used herein refer to the orientation of the respiratory protection device as shown in FIGS. 1 and 7.

An upright supporting bar 15, shown in FIGS. 2 to 4, extends along the minor axis of the outlet 9. FIG. 1 shows a non-return valve in the form of a flap valve 17 secured to the supporting bar 15 by means of a protrusion 19 extending from a central region of the supporting bar and passing through an aperture in the flap valve. As shown in FIG. 4, the protrusion 19 has a domed surface and a flat under surface. The flap valve 17 is made of a resilient material, such as an elastomeric material, and the domed surface of the protrusion 19 allows the protrusion to pass through the aperture in the valve, while the flat under surface retains the valve in position. The flap valve 17 is positioned within the outlet 9 with a periphery of the valve against a shoulder 21 which is provided around an inner circumference of the outlet 9 (see FIG. 4). The shoulder 21 prevents the periphery of the valve from being drawn completely through the outlet 9 and into the housing 1 during use.

As an alternative to the protrusion 19, the flap valve 17 could be secured to the supporting bar 15 by other suitable means, such as an adhesive.

As shown in FIGS. 3 and 5, the central elliptical region of the housing 1 is provided with engagement members 23 which extend inwardly from upper and lower regions of an inner face of the forward surface. The engagement members have inclined surfaces to facilitate the mounting of the mouthpiece 5 with the housing 1 as explained hereinafter.

As shown in FIGS. 3 and 5, the rearward edge of the curved forward surface 7 forms a second surface 25 opposing the first surface 7 which in use is positioned adjacent to the face of a user. Retaining members 27, in the form of lips extending inwardly parallel to the plane of the second surface 25, are provided on upper, lower and side portions of the second surface and are adapted to retain the foam elements 3 within the chamber of the housing 1 as will be explained hereinafter.

Resilient flexible securing members 29 are provided on upper and lower portions of the second surface 25 in a region opposite the outlet 9 in the front surface and are adapted to retain a portion of the mouthpiece within the chamber of the housing 1 as will be explained hereinafter.

The foam elements 3 are open cell polymeric foam elements which permit the passage of air therethrough. A first foam element is dimensioned to correspond to the shape of the first side portion of the chamber of the housing 1 and a second foam element is dimensioned to correspond to the shape of the second side portion of the chamber of the housing 1.

The foam elements are interference fitted into the side portions of the chamber of the housing 1 between the forward surface of the housing and the retaining members 27 provided on the upper, lower and side portions of the second surface 25 of the housing 1. The elements are retained in position within the side portions of the chamber by the retaining members 27.

As shown in FIG. 5, each foam element has an inwardly curved edge 31 which corresponds to the shape of the outlet 9 in the front surface of the housing 1. The inwardly curved edges of the foam elements 3 ensure that the foam elements do not extend across the outlet 9 in the central region of the housing 1.

A suitable filter material (not shown) of any well known form is laminated to the open faces of the foam elements 3, That is, the filter is laminated to the faces of the foam elements that are nearest to the user in use. The filter material may be a conventional untreated web of filter material. Alternatively, the filter material may comprise one or more of: an electrostatic filter material, a filter material incorporating activated carbon, a filter material incorporating an open cell foam incorporating activated carbon, and a filter material incorporating an open cell foam coated with a hygroscopic substance.

The mouthpiece 5 is substantially elliptical in cross-section with the major axis of the ellipse being substantially horizontal. The mouthpiece 5 is formed with an outwardly flared (plate-like) mouth portion 33, which is shaped and adapted to be received in the mouth of the user between the lips and teeth of the user, and an elliptical tube 35, open at each end, extending away from a central region of the mouth portion 33. The mouthpiece 5 is substantially held in place in the mouth of a user by pressure against the inside of the user's lips from the plate-like mouth portion 33, with the teeth acting as a fulcrum against which the mouth portion presses. This arrangement minimises the active effort required by the user to maintain the mouth portion of the mouthpiece in position within the mouth.

As shown in FIG. 6, the tube 35 passes through a wall member 37, which is provided at a slight angle from a plane perpendicular to the longitudinal axis of the tube, and extends outwardly therefrom. The wall member 37 is angled such that an upper region 39 of the wall member is further from the mouth portion 33 than a lower portion 41 of the wall member.

Shoulders 43 are provided on forward portions of the upper region 39 and the lower region 41 of the wall member 37. The shoulders 43 are used, in conjunction with the resilient, flexible securing members provided on the upper and lower portions of the second surface 25, as described hereinbefore, to secure a forward portion 45 of the mouthpiece 5 to the housing 1. The securing members flex to pass over the shoulders 43 of the wall member 31 and snap fit behind the shoulders. The angle of the wall member 37 described hereinabove serves to present the shoulders 43 in the correct position relative to the securing members 29 to facilitate securing together thereof.

The forward portion 45 of the tube 35, extending outwards from the wall member 37 is provided with four gaps 47 through the annular wall of the tube, two gaps each side of an upper portion 49 and a lower portion 51 of the forward portion 45 of the tube 35. The two gaps 47 on each side of the forward portion 45 of the tube 35 are separated by a central bar 53 which serves to increase the rigidity of the forward portion 45 of the tube.

The gaps 47 in the forward portion 45 of the tube 35 have a total area larger than the cross-sectional area of an elliptical bore of a portion of the tube provided between the wall member 37 and the mouth portion 33. Preferably, the total area of the gaps is approximately 20 percent larger than the cross-sectional area of the portion of the tube 35 between the wall member 37 and the mouth portion 33. The difference in area is to compensate for the fact that in use there is more resistance to air flow through the gaps 47 than through the bore of the tube as the gaps are in contact with the foam elements as will be described hereinafter.

The mouthpiece 5 and the outlet 9 in the housing 1 are substantially coaxial so as to allow substantially unobstructed passage of air from the mouthpiece 5 to the outlet 9 when the user exhales.

The forward portion 45 of the mouthpiece 5 is attached to the housing 1 by the upper portion 49 and lower portion 51 of the front portion of the mouthpiece being push fitted between the engagement members 23, on the inner face of the front surface, and the upper and lower inner faces of the housing 1 (see FIG. 3). The engagement members 23 and the upper portion 49 and lower portion 51 have complementary inclined surfaces which, when fitted together, produce a wedging action to retain the surfaces relative to each other.

The forward portion 45 of the mouthpiece 5 is secured relative to the housing by the securing members 29 provided on the upper and lower portions of the second surface, as described hereinbefore, which are snap fitted behind the shoulders 43 provided on the upper and lower regions 39, 41 of the wall member 37 as described hereinbefore.

The combination of the securing members 29 securing the wall member 37 to the housing and the engagement members 23 attaching the front portion of the mouthpiece to the housing substantially prevents the mouthpiece 5 from moving or rotating relative to the housing 1.

When the mouthpiece 5 is secured to the housing 1, the forward portion 45 of the mouthpiece is positioned between the curved edges 31 of the foam elements 3 and the wall member 37 applies pressure on the foam elements such that the curved edges 31 are urged towards the gaps 47 in the forward portion 45 of the mouthpiece 5, effectively sealing the foam elements 3 to the forward portion of the mouthpiece. The wall member also retains the curved edges of the foam elements within the housing.

In use of the respiratory protection device according to the present invention, the mouth portion 33 of the mouthpiece 5 of the device is inserted into the user's mouth between the lips and the teeth and in this position gives rise to relatively little saliva build-up, allows clenching of the teeth and does not inhibit swallowing as compared with a conventional mouthpiece which is positioned with lugs between the teeth of the user, making swallowing difficult. Further, the device according to the present invention does not cover the nose of the user because it has been found that above relatively low levels of exercise substantially all inspiration takes place through the mouth.

The portion of the tube 35 of the mouthpiece between the wall member 37 and the mouth portion 33 is of sufficient length that the second surface 25 of the housing is positioned adjacent to, but distanced from, the face of the user.

During expiration the flap valve 17 flexes to allow exhaled air to pass through the outlet 9 with little or no resistance, while during inhalation the valve 17 is drawn against the shoulder 21 of the outlet in the housing 1 to seal therewith and air is therefore drawn into the chamber and through the foam elements 3 via the filter. The inhaled air passes through the gaps in the mouthpiece and onward to the user. Unwanted particles, for example produced by internal combustion engines, entrained in the inhaled air are entrapped by the filter and the foam elements 3 and consequently are not inhaled by the user.

If the filter needs to be changed for any reason, this can be readily accomplished simply by removing the mouthpiece 5 from the housing 1, removing the foam elements with the filters attached, replacing the used foam elements with new ones, and replacing the mouthpiece.

The filter material is positioned on that side of the housing 1 adjacent to the face of the user. That is, the filter is between the foam elements 3 and the face of the user.

The filter material is not obstructed in any way, for example due to contact with the face of the user, and the entire surface area of the filter material is therefore able to pass air into the foam elements 3 in the chamber and to the mouthpiece 5 with very little obstruction and therefore without applying any significant stress to the inspiratory muscles of the user thereby minimising the additional work of breathing.

It should be appreciated that although a filter laminated to the foam elements has been described, a respiratory protection device in accordance with the present invention could have a filter which is separate from, but releasably attached to, the foam elements. Alternatively, a respiratory protection device in accordance with the present invention need not have the filter. The foam elements 3, could be used to filter material, for example particulate material, from the inhaled air.

FIGS. 7 to 10 show a second embodiment of a respiratory protection device in accordance with the present invention.

Similar features to those in the first embodiment have been given corresponding reference numbers.

The respiratory protection device comprises a housing 1, a pair of porous elements 3 and a communicating member in the form of a mouthpiece 5.

The housing 1 is in the form of a chamber which is generally U-shaped in cross-section forming top and bottom walls of the housing with a (first) forward surface 7 therebetween. An elliptical cross-sectioned portion of the housing, the major axis of the ellipse being substantially horizontal, is provided in a central region of the forward surface of the housing 1. A pair of wings 11 extend laterally, one from each side of the central elliptical region, forming a first side portion and a second side portion of the chamber. Each wing 11 is curved so as to extend in use around the face of the user and the outer ends of the wings are closed at 13 forming side walls of the chamber. A plurality of apertures 55 is provided through the forward surface of the housing 1.

As shown in FIG. 9, the central elliptical region of the housing 1 is provided with engagement members 23 which extend inwardly from upper and lower regions of an inner face of the forward surface and are arranged to enable the mouthpiece 5 to be attached to the housing 1 as explained hereinbefore in regard to the embodiment shown in FIGS. 1 to 5.

The rearward edge of the curved forward surface forms a second surface 25 opposing the first surface 7 which in use is positioned adjacent to the face of a user. Retaining members 27 are provided on upper, lower and side portions of the second surface and are adapted to retain the foam elements 3 within the chamber of the housing 1 as explained hereinbefore in regard to the embodiment shown in FIGS. 1 to 5.

Securing members 29 are also provided on upper and lower portions of the second surface 25 in a region opposite the outlet 9 in the front surface and are adapted to retain a forward portion 45 of the mouthpiece 5 within the chamber of the housing 1 as explained hereinbefore in regard to the embodiment shown in FIGS. 1 to 5.

The porous elements 3 are in the form of open cell polymeric foam elements so as to permit the passage of air therethrough. As for the embodiment shown in FIGS. 1 to 6, the foam elements are configured to correspond to the shape of the side portions of the chamber of the housing 1 and have inwardly curving edges 31.

The foam elements 3 are impregnated with a passive heat and moisture exchange material, for example substantially 16 cubic centimetres per element, in the form of a hygroscopic material. The hygroscopic material picks up moisture from air exhaled through the foam elements and returns it to air inhaled through the foam elements. The provision of warm and humidified air to the user prevents drying of airways of the user, which can be a trigger for asthma.

An important contributor to the efficiency of the return of moisture to the inhaled air is the temperature of the inhaled air. The positioning of the impregnated foam elements 3 in the housing 1 relative to the face of a user enables exhaled heat to be retained and for the hygroscopic material to absorb radiant heat from the user's face. The heat retained by the foam elements serves to heat inhaled air and thus enhance the return of moisture thereto.

The foam elements are fitted and retained within the side portions of the chamber of the housing 1 as explained hereinbefore in regard to the embodiment shown in FIGS. 1 to 5.

The mouthpiece 5 is substantially identical to that shown in FIG. 6, and is fastened relative to housing 1 and the foam elements 3 as described hereinbefore in regard to the embodiment shown in FIGS. 1 to 5.

In use of the respiratory protection device according to the present invention, the mouth portion 33 of the mouthpiece 5 of the device is inserted into the user's mouth between the lips and the teeth and in this position gives rise to relatively little saliva build-up, allows clenching of the teeth and does not inhibit swallowing as compared with a conventional mouthpiece which is positioned with lugs between the teeth of the user, making swallowing difficult. Further, the device according to the present invention does not cover the nose of the user because it has been found that above relatively low levels of exercise all inspiration takes place through the mouth.

During expiration, the majority of exhaled air passes down the tube 35 of the mouthpiece 5 and into the foam elements 3, via the gaps 47 in the tube 35 of the mouthpiece 5, prior to being emitted from the chamber of the housing 1 in the region of the user's face. A minor quantity of exhaled air passes through the foam elements and out of the housing via the plurality of apertures 55 through the forward surface of the housing 1.

During inhalation, the majority of air is drawn into the foam elements 3 through the open faces of the foam elements positioned adjacent to the user's face. However some air is drawn in via the plurality of apertures 55 through the front surface of the housing 1. Unwanted particles, for example produced by internal combustion engines, entrained in the inhaled air may be entrapped by the foam elements 3 and as such are not inhaled by the user.

If, for any reason the foam elements 3 need to be changed this can be readily accomplished simply by removing the mouthpiece 5 from the housing 1, removing the foam elements, replacing the used foam elements with new ones, and replacing the mouthpiece.

It should be appreciated that the respiratory protection device shown in FIGS. 7 to 10 could incorporate additional features from the respiratory protection device shown in FIGS. 1 to 5, and vice versa.

The components of a respiratory device in accordance with the present invention may be made of any suitable material. For example, the housing 1 may be made of a thermoplastic plastics material, preferably polypropylene. Alternatively, the housing 1 may be made from a flexible material that provides protection to the user's mouth in the case of an impact. The mouth portion 33 of the mouthpiece 5, at least, should be relatively flexible in order to be comfortable in use and not to present a hazard in the event of an accident. Therefore, the mouth portion 33 may be a silicone material. Flavouring may be added to the mouth portion 33 of the mouthpiece to mask any taste that might be associated with the material forming the mouth portion.

If desired, a respiratory device in accordance with the present invention may be provided with a cord which will in use extend loosely around the neck of the user in order that the user does not need to hold the device when it is not in use. In this way, the device can readily be taken out and replaced, for example when waiting at traffic lights.

Aromatherapy oils, known to a person skilled in the art, could be incorporated into the foam elements 3 to fragrance and flavour the inhaled air.

It should also be appreciated that the chamber of a respiratory device in accordance with the present invention may only extend laterally to one side of the mouthpiece.

Although a communicating member 5 has been described hereinbefore as a mouthpiece comprising a mouth portion 33, which is shaped and adapted to be received in the mouth of the user between the lips and teeth of the user (see FIG. 6), it should be appreciated that the communicating member could alternatively be provided with a mask-like portion 57 which is shaped and adapted to be positioned over the mouth, and possibly the nose, of the user (see FIG. 11), if the use of a mask-like portion is preferred by the user over a mouth portion.

The mask-like portion 57 of the communicating member 5 is provided with means 59 for securing the communicating member to the head of the user, for example a strap, preferably an elasticated strap. The communicating member 5 with the mask-like portion 57 is secured and retained in position relative to the housing as described hereinbefore in regard to the embodiments shown in FIGS. 1 to 5 and FIGS. 7 to 10.

The disadvantages of known face masks are minimised in the embodiment of the present invention comprising the mask-like portion by the housing containing the foam elements not being integral with the mask-like portion.

1. A respiratory protection device comprising:
a housing (1) defining a chamber within the housing, the chamber being open in a region which in use is adapted to be nearest to a user;
a porous element (3) within the chamber the porous element being dimensioned to correspond to the shape of the chamber within the housing (1): and a communicating member (5) provided with a tubular element (35) provided with a portion (45) having gaps (47) through an annular wall (37) adapted to communicate with the porous element within the chamber, the portion (45) being positioned between curved edges (31) of the porous element (3), and the annular wall (37) applying pressure on the porous element (3) such that the curved edges (31) are urged towards the gaps (47) of the communicating member (5) so as to seal the porous element (3) to the portion (45) of the communicating member and to retain the curved edges (31) of the porous element (3) within the housing (1).

2. A device as claimed in claim 1, wherein the porous element (3) comprises a foam material.

3. (canceled)

4. A device as claimed in claim 1, wherein the porous element (3) incorporates a hygroscopic material.

5. A device as claimed in claim 1, wherein a filter is incorporated in the porous element (3).

6. A device as claimed in claim 5, wherein the filter comprises a web of filter material mounted on the porous element (3).

7. A device as claimed in claim 5, wherein the filter comprises a layer of activated carbon material.

8. A device as claimed in claim 1, wherein the communicating member (5) is provided with a plate-like portion (33) adapted to be received in the mouth of the user.

9. A device as claimed in claim 8, wherein the communicating member (5) is adapted to be received behind the lips and in front of the teeth of the user.

10. A device as claimed in claim 1, wherein the communicating member (5) is provided with a mask-like portion (57) adapted to be worn over the mouth of the user.

11. (canceled)

12. (canceled)

13. A device as claimed in claim 1, wherein the total area of the gaps (47) is greater than a cross-sectional area of the tubular element (35) defined by the annular wall (37).

14. A device as claimed in claim 13, wherein the total area of the gaps (47) is substantially 20 percent greater than the cross-sectional area of the tubular element (35) defined by the annular wall (37).

15. (canceled)

16. A device as claimed in claim 1, wherein the wall portion (37) of the communicating member (5) is provided with means (43) adapted to secure the wall portion of the communicating member to securing members (29) of the housing (1).

17. A device as claimed in claim 16, wherein the securing means comprise shoulders (43).

18. A device as claimed in claim 17, wherein the securing members (29) of the housing (1) are of resilient material adapted to fit over the shoulders (43) of the wall portion (37) of the communicating member (5) thereby securing the member to the housing.

19. A device as claimed in claim 1, wherein the housing (1) is provided with first and second opposing surfaces (7, 25).

20. A device as claimed in claim 19, wherein an outlet (9) is provided in the first surface (7) of the housing (1).

21. A device as claimed in claim 20, wherein a non-return valve (17) is mounted across the outlet (9) in the first surface (7).

22. A device as claimed in claim 21, wherein the non-return valve (17) comprises a flap valve.

23. A device as claimed in claim 20, wherein the outlet (9) is substantially coaxial with the communicating member (5).

24. A device as claimed in claim 23, wherein the cross-sectional area of the outlet (9) corresponds substantially to the cross-sectional area of the tubular element (35) of the communicating member (5).

25. A device as claimed in claim 19, wherein a plurality of apertures (55) are provided in the first surface (7) of the housing (1) adapted to permit air to pass through the first surface during inhalation and exhalation by the user.

26. A device as claimed in claim 19, wherein a retaining member (27) is provided on the second surface (25) of the housing (1) adapted to retain the porous element (3) within the chamber of the housing.

27. A device as claimed in claim 26, wherein the retaining member (27) comprises an extending lip.

28. A device as claimed in claim 1, wherein the chamber extends laterally to at least one side of the communicating member (5).

29. A device as claimed in claim 1, wherein the housing (1) is curved so as in use to extend around the face of the user.
